(19) United States
(12) Patent Application Publication
(10) Pub. No.: US 2015/0315162 A1
(43) Pub. Date: Nov. 5, 2015

(54) Process for producing fucoxanthinol extract and methods of use

(71) Applicants: Jean Mamelona, Moncton (CA); Denis M. Brion, Montreal (CA)

(72) Inventors: Jean Mamelona, Moncton (CA); Denis M. Brion, Montreal (CA)

(21) Appl. No.: 14/120,163

(22) Filed: May 1, 2014

(51) Int. Cl. C07D 301/32; A61K 31/336

(52) U.S. Cl. CPC C07D 301/32; A61K 31/336

(57) ABSTRACT

A process for obtaining a purified fucoxanthinol extract from an Echinozoa tissue or organ is disclosed, as well as methods of using said extract. Compositions comprising the extract, and their uses, are also described. Uses of the purified extract include methods for making medicaments or treatments for conditions such as, but not limited to, obesity, cancer, oxidative stress, diabetes, neurodegenerative disorders, and high blood pressure.

This application claims the benefit of U.S. Provisional Application 61/854,940, filed May 6, 2013.

### TECHNICAL FIELD

The present invention generally relates to extracts obtained from marine invertebrates. More specifically, the present invention relates to a method of extracting fucoxanthinol compositions from Echinoidea tissues/organisms, and the compositions extracted by using the method.

### BACKGROUND ART

Carotenoid compounds have been studied intensively due to their potential medicinal applications. In particular, some carotenoid compounds have been shown to display significant anti-oxidative activity and free radical elimination characteristics, as well as the ability to inhibit the growth of cancer. Oxygen-free carotenoids are known as carotenes, while those carotenoids that contain oxygen are known as xanthophylls.

One xanthophyll that has received significant attention is fucoxanthin, which is derived from brown algae. Fucoxanthin is a pigmented compound in the chloroplast of brown algae and seaweeds that give the plants their characteristic colour. When fucoxanthin is ingested, it is hydrolyzed into the deacetylated form, fucoxanthinol. Studies in mice indicate that following ingestion of fucoxanthin, it is metabolized into fucoxanthinol in the digestive tract, and then further converted to amarouciaxanthin A in the liver (Hashimoto et al., 2009).

Research on fucoxanthin and fucoxanthinol has demonstrated a diverse range of activity. As with other carotenoids, fucoxanthin and its metabolites have been shown to have anti-oxidative effects—including free radical scavenging activity and singlet oxygen quenching. (Sachindra et al., 2007). In addition, fucoxanthin has shown anti-inflammatory effects through the inhibition of inducible nitric oxide synthase and cyclooxygenase 2 protein expression, as well as reducing levels of nitric oxide, prostaglandin E2, tumor necrosis factor-□ and interleukins 1β and 6 (Heo et al. 2008 and Kim et al., 2010). Reactive oxygen species generated as a result of UV radiation exposure were also decreased as a result of fucoxanthin (Heo and Jeon, 2009) suggesting that fucoxanthin may have a protective effect for the skin.

Fucoxanthin and fucoxanthinol also have anti-obesity effects. For example, fucoxanthin has been combined with pomegranate seed oil with the combination having the effect of reducing body and liver fat content and improving liver function, as well as increasing resting energy expenditure (Maeda et al., 2005; Abidov et al., 2010). Further research suggests that the anti-obesity effects of fucoxanthin and its metabolites may stem from the regulation of expression of the uncoupling protein (UCP1) in white adipose tissue (WAT), which caused a decrease of abdominal fat in both rats and mice. (Miyashita et al., 2011) Maeda et al. also successfully demonstrated that fucoxanthin decreases blood glucose levels in diabetic mice, in addition to reducing the levels of insulin in plasma.

Fucoxanthin and fucoxanthinol have been shown to induce apoptosis in a number of human cancer cell lines, including those for prostate and colon cancers, as well as leukemia. While the full understanding of the effects of fucoxanthinol on cancer cells is yet to be elucidated, it has been reported that fucoxanthinol down regulates the peroxisome proliferator-activated receptor □, which also contributes to its activity as an anti-obesity compound. (Hosokawa et al. 2004) With regard to cancer treatment, fucoxanthin has been shown to inhibit metastatic potential of cancer cells (Chung et al., 2013) in in vitro and in vivo models. Fucoxanthinol also inhibits the cell viability in T-cell lines infected with human T-cell leukemia virus type 1, and adult T-cell leukemia. The fucoxanthinol induces apoptosis by arresting the cell cycle during the G1 phase (Ishikawa et al., 2008). Fucoxanthin treatment has reduced viability of three lines of human prostate cancer cells, also through apoptosis (Kotake-Nara et al. 2001). Fucoxanthinol has been shown to exert a stronger effect than fucoxanthin in the antiproliferation of prostate cancer cells (Asai et al., 2004) and shows almost twice the efficacy of fucoxanthin in the inhibition of HTLV-1-infected T-cell lines and ATL cells. (Ishikawa et al. 2008). A recent review of the anti-cancer effects of fucoxanthin and fucoxanthinol outlines the various mechanisms through which these compounds exert their influence (Ravi Kumar et al, 2013).

In addition, fucoxanthin and fucoxanthinol have been shown to possess antiangiogenic activity, preventing the development of new blood vessels, which can be useful in the inhibition of tumour growth. (Sugawara et al., 2006)

Fucoxanthin can be chemically synthesized, but the process is expensive and the yield is low. Accordingly, it is desirable to find a way to extract fucoxanthinol from natural sources in a cost effective method with an appropriate yield such that the compound can be used in compositions for medicinal or health purposes. Previous work has shown that fucoxanthin can be extracted from marine diatoms (Kim, 2014) by a solvent extraction method. Different solvent/diatom ratios and different temperatures resulted in different yields ranging up to 0.78% fucoxanthin. High pressure solvent extraction of fucoxanthin from seaweed (Eisenia bicyclis) gave maximum extraction results of 0.42 mg/g (Shang et al., 2011). Quintain et al. (2013) showed that fucoxanthin can be extracted from seaweed (Undaria pinnatifida) using a supercritical carbon dioxide method with a range of temperatures and pressures, as well as flow rates, resulting in extraction rates of 0.53 to 1.22 g extract per 100 g sample. These processes do not encompass the extraction of fucoxanthinol.

In order to extract fucoxanthinol, which is more active than fucoxanthin, it is necessary to extract it from organisms that digest the seaweeds and produce the metabolite in the digestive tract.

Fucoxanthinol can be extracted from sea squirt (Ascidian) or other Echinozoa tissues as outlined in U.S. Pat. No. 8,445,028 using a method that combines tissues with a dispersing agent and then uses a solvent extraction process, followed by high pressure liquid-solid extraction. Other methods of fucoxanthinol extraction that are simpler and result in appropriate yields are desirable and useful for the preparation of medications using fucoxanthinol.

The present description refers to a number of documents, the content of which is herein incorporated by reference in their entirety.

### SUMMARY OF THE INVENTION

In the studies described herein, it is shown that fucoxanthinol extracts may be obtained from various organs and tissues of marine invertebrates belonging to the Echinozoa subphylum.

In one aspect, the present invention provides an Echinozoa tissue or organ extract obtained by a process comprising:
providing Echinozoa tissues or organs;
mixing or blending the tissues or organs to achieve homogenization;
using solvent extraction to extract the fucoxanthinol from the blended tissues or organs;
optionally centrifuging the solvent-tissue mixture to remove particulate matter;
evaporating a portion of the liquid component to remove solvent and water;
filtering the remaining liquid; and
evaporating the final filtered liquid to remove the majority of the water and solvent in order to achieve a purified fucoxanthinol extract.

In one aspect, the Echinozoa tissue and or organs are provided in a frozen state and thawed prior to homogenization.

In another aspect, the above-mentioned extraction process is performed at a temperature of about 15° C. to about 25° C. In a further aspect, the evaporation step in the above-mentioned extraction process is performed at a temperature of about 35° C. to about 50° C.

In another aspect, the above-mentioned tissues or organs are (i) tissues or organs of the digestive tract, (ii) viscera, or (iii) a combination of (i) and (ii).

In another aspect, the present invention provides a use of (i) the above-mentioned extract, (ii) the above-mentioned composition, or (iii) a combination of (i) and (ii), as a medicament.

In another aspect, the present invention provides a use of (i) the above-mentioned extract, (ii) the above-mentioned composition, or (iii) a combination of (i) and (ii), for the preparation of a medicament.

In another aspect, the present invention provides a composition comprising the above-mentioned extract and a carrier. In various aspects of the present invention, the above-mentioned composition is an anti-oxidant composition an anti-obesity composition, an anti-cancer composition, or an anti-angiogenic composition. In other aspects, the above-mentioned composition is a composition that reduces blood glucose or increases resting energy expenditure.

In another aspect, the present invention provides a use of (i) the above-mentioned extract, (ii) the above-mentioned composition, or (iii) a combination of (i) and (ii), for prevention or treatment of obesity or cancer.

In another aspect, the present invention provides a use of (i) the above-mentioned extract, (ii) the above-mentioned composition, or (iii) a combination of (i) and (ii), for the preparation of a medicament for (a) decreasing or inhibiting oxidative stress in a cell or tissue, (b) prevention or treatment of a condition associated with oxidative stress, (c) reducing blood pressure, (d) increasing energy expenditure rate, (e) reducing inflammation, (f) treating a cancerous condition, or (f) treatment of a condition requiring a neuroprotective effect.

In another aspect, the present invention provides a method for preventing or treating a condition, said method comprising administering to said subject (i) the above-mentioned extract, (ii) the above-mentioned composition, or (iii) a combination of (i) and (ii).

Other objects, advantages and features of the present invention will become more apparent upon reading of the following non-restrictive description of specific embodiments thereof, given by way of example only with reference to the accompanying drawings.

### BRIEF DESCRIPTION OF THE DRAWING

FIG. 1 shows a UV chromatogram for a representative sample of the extract with peaks for fucoxanthinol at RT 9.51 and for fucoxanthin at RT 11.50.

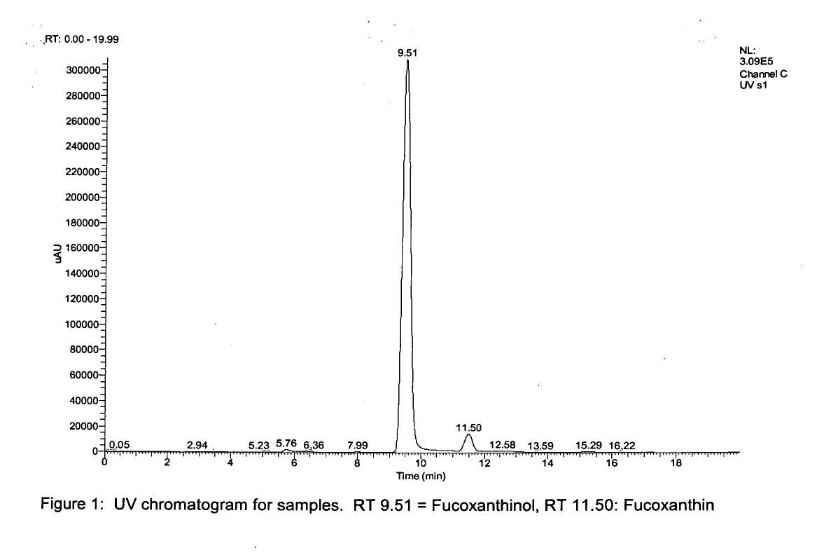

### DETAILED DESCRIPTION OF THE PREFERRED EMBODIMENT OF THE PRESENT INVENTION

The present invention relates to fucoxanthinol extracts obtained from the tissues of organisms in the Echinodermata phylum, specifically in the Echinozoa subphylum. Echinozoa are invertebrate marine animals found at all ocean depths. As a group, the Echinozoa are known to eat algae and seaweed species, including those algae and seaweed in the brown colour category, which contain fucoxanthin. When the Echinozoa eat these algae and seaweed species, their digestive processes metabolize the fucoxanthin to fucoxanthinol.

The processing of various sea urchin species for food purposes is designed to remove the edible roe from the spiny exterior of the urchin, as well as separating the roe from the gut portion of the organism. The gut of the urchin, including the digestive system, is not typically eaten or used for other purposes. As a result, sea urchin processing facilities typically dispose of the sea urchin gut as a “waste product” which is sent directly to dumping sites. The present invention uses this former “waste product” for the extraction of fucoxanthinol.

In some embodiments, the present invention uses the tissues of Echinozoa from the Diadematoidea, Arbacioida and Echinoida orders. In another embodiment, the tissues of Echinozoa from the Strongylocentrotidae family are used. In one embodiment, the tissues of the sea urchin Strongylocentrotus droebachiensis (Green Sea Urchin) are used. S. droebachiensis is widespread on the northern hemisphere, and has also been harvested by man as a food source for thousands of years.

Accordingly, in a first aspect, the present invention provides a process for preparing an Echinozoa tissue or organ extract comprising:
providing Echinozoa tissues or organs;
mixing or blending the tissues or organs to achieve homogenization;
using solvent extraction to extract the fucoxanthinol from the blended tissues or organs;
optionally centrifuging the solvent-tissue mixture to remove particulate matter;
evaporating a portion of the liquid component to remove solvent and water;
filtering the remaining liquid; and
evaporating the final filtered liquid to remove the majority of the water and solvent in order to achieve a purified fucoxanthinol extract.

In some cases, it is desirable that the Echinozoa tissue and or organs are provided in a frozen state. The processing of a large quantity of organisms results in large volumes of tissue and organs that may need to be stored or transported prior to the extraction process. In order to prevent the decomposition of the tissues and the potential reduction of fucoxanthinol yield, the tissues and organs may be frozen in large quantities at the source, such as a processing factory. Prior to the blending or homogenizing step, the tissues must be thawed in order to facilitate the homogenization. As will be understood by those of skill in the art, different quantities of frozen tissue must be thawed for longer or shorter periods of time depending on the amount of tissue and the size of the frozen mass of tissue, in order that the tissue throughout the mass is thawed.

The starting material (i.e. Echinozoa tissue or organ) is homogenized using commercially-available grinders, blenders or comparable devices. For example, complete homogenization may be achieved at a minimum of 2500 rpm in an industrial blender in order to optimize the eventual fucoxanthinol yield. The homogenization of the tissue allows for the maximum surface area exposure, thus maximizing the yield of fucoxanthinol that is obtained from the extraction process. In an embodiment, the starting material is received in a frozen condition, and is subsequently thawed by leaving the material at room temperature before being ground and homogenized. The ground/homogenized material may be stored, preferably in a frozen state, before extraction. In an embodiment, the ground/homogenized material is kept frozen. (e.g., at about −20° C.) before extraction. One of skill in the art will recognize that the ideal homogenization speed and duration will depend upon the quantity of tissue being homogenized.

The extraction process is performed in the presence of a solvent, such as an alcohol or polyol (or any combination/mixture thereof). Solvents that may be used for the extraction process include, for example, ethanol, methanol, propanol, n-hexane and acetone. The solvents may be pure (100%) solvent or may be mixed with water (for example, 80% ethanol may be used). The homogenized material is combined with the solvent at a 1:1 v/v ratio and thoroughly mixed for a period of time. As one of skill in the art will realize, the length of time necessary for the mixing will depend upon the quantity of material. For example, 50 kg of homogenized tissue is allowed to mix for 8 to 12 hours per extraction. Following the mixing phase, the slurry is left to settle in order to separate the solvent containing the fucoxanthinol from the solid homogenized tissue material. In an embodiment of the present invention, the above-mentioned extraction process is performed at a temperature of about 15° C. to about 25° C. In a further embodiment, the evaporation step in the above-mentioned, extraction process is performed at a temperature of about 35° C. to about 50° C.

Following the settling of the material, the liquid portion containing the solvent and extracted fucoxanthinol is separated from the solid material. A large portion of the liquid portion may be removed by pumping or decanting in a way that minimizes mixing with any settled or suspended solids. Fresh solvent is added to the remaining slurry for subsequent extractions. One of skill in the art will recognize that extractions may be performed multiple times in order to maximize the yield of fucoxanthinol. For example, extractions may be performed from one to five times in order to achieve a good yield.

The liquid portion that is removed from the slurry may be partly clear, but a portion of it may have a high concentration of suspended solids. This portion is likely to be the portion that was closest to the settled material. Therefore, a centrifugation step is ideally used to eliminate any such suspended solids from the liquid. Centrifugation may be performed on a suitably sized centrifuge depending on the amount of liquid requiring centrifugation, and should be performed at a speed of 2500 to 6500 rpm for 5 to 20 minutes.

The extract obtained from the top of the slurry, and the liquid from the centrifugation step is in the form of a liquid concentrate. The concentrate may be further concentrated (e.g., evaporation of the extraction solvent and water portion of the concentrate) using apparatus and methods well known in the art, to obtain a solid or dry extract. In an embodiment, the liquid extract is dried via gaseous flow, e.g., under the flow of an inert gas, e.g., under (e.g., continuous) nitrogen flow. In another embodiment, the liquid extract is dried via evaporation, e.g., using a rotating evaporator or similar device. In another embodiment, the liquid extract is dried at ambient temperature (e.g., between about 15° C. to about 50° C., and more particularly between about 35° C. to about 50° C.) In addition, the evaporation may be performed under vacuum, such as a vacuum with a value between 72 and 35 mbar. Ideally, the water and solvent is evaporated at a rate between 3 and 60 L per hour.

In order to maximize purity of the final fucoxanthinol extract, a filtration step may be used before the final evaporation step. The liquid from the first evaporation may be filtered to remove any suspended solids or precipitated proteins. Filtering may be conducted with a filter having a pore size of about 22 μm to about 100 μm.

Following filtration, a final evaporation step is used to produce the oily extract containing fucoxanthinol. The final evaporation is similar to the first evaporation, and should be stopped when no further water or solvent is being obtained, at which point less than 10% of water or solvent is left in the oil.

In another aspect, the present invention provides an Echinozoa tissue or organ extract containing fucoxanthinol obtained by the above-mentioned process.

In another aspect, the present invention provides a composition or formulation (e.g., an antioxidant composition or formulation) comprising the above-mentioned extract and a carrier or excipient (e.g., a pharmaceutically acceptable, cosmetically acceptable, or a consumable carrier/excipient).

In various embodiments, an extract of the invention may be used therapeutically in formulations or medicaments to prevent or treat a condition, such as a condition associated with oxidative stress. “Oxidative stress” as used herein generally refers to oxidative damage in a cell, tissue, or organ, caused by reactive oxygen species (ROS), such as free radicals and peroxides. The level of oxidative stress is typically determined by the balance between the rate at which oxidative damage is induced and the rate at which it is efficiently repaired and removed. Other conditions which may be prevented or treated using an extract of the invention include obesity and those forms of cancer which can be treated or inhibited through the use of fucoxanthinol. In addition, the fucoxanthinol extract of the invention may be used therapeutically to increase resting energy expenditure, to reduce blood glucose levels, and as an antiangiogenic compound.

The invention provides corresponding methods of medical treatment, in which a therapeutic dose of an extract of the invention is administered in a pharmacologically acceptable formulation, e.g. to a patient or subject in need thereof. Accordingly, the invention also provides therapeutic compositions comprising an extract of the invention and a pharmacologically acceptable excipient or carrier.

A “therapeutically effective amount” or “effective amount” (in the context of treatment) refers to an amount effective, at dosages and for periods of time necessary, to achieve the desired therapeutic result, such as a reduction of oxidative stress and in turn a reduction in progression of or the amelioration of an associated condition. A therapeutically effective amount may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” or “effective amount” (in the context of prevention) refers to an amount effective, at dosages and for periods of time necessary, to achieve the desired prophylactic result, such as preventing or inhibiting the rate of disease onset or progression of a condition associated with oxidative stress. A prophylactically effective amount can be determined as described above for the therapeutically effective amount. For any particular subject, specific dosage regimens may be adjusted over time according to the individual need and the professional judgement of the person administering or supervising the administration of the compositions.

Various embodiments of the present invention provide for the treatment of obesity, oxidative stress, cancer, diabetes and angiogenesis, comprising the step of administering to a mammal a therapeutically effective amount of the fucoxanthinol extract obtained by the extraction method of the invention.

The extract or composition of the present invention may be administered in any number of conventional dosage forms, e.g., topical, oral, parenteral, rectal, transdermal, and the like. Oral or rectal dosage forms include capsules, tablets, pills, powders, cachets and suppositories. Liquid oral dosage forms include solutions and suspensions. Parenteral preparations include sterile solutions and suspensions. Topical dosage forms can be creams, ointments, lotions, transdermal devices and the like. Except insofar as any conventional media or agent is incompatible with an extract of the invention, use thereof in the pharmaceutical compositions of the invention is contemplated. Supplementary active compounds can also be incorporated into the compositions.

The formulations and pharmaceutical compositions contemplated by the above dosage forms can be prepared with conventional pharmaceutically acceptable excipients and additives using conventional techniques. Such pharmaceutically acceptable excipients and additives are intended to include carriers, binders, flavorings, buffers, thickeners, color agents, stabilizing agents, emulsifying agents, dispersing agents, suspending agents, perfumes, preservatives, lubricants, etc.

Further, the composition can be prepared such that an extract of the invention can be administered in a controlled or time release formulation, for example in a composition which includes a slow release polymer for prolonged release.

Suitable pharmaceutically acceptable carriers include solvents, coatings, dispersion media, and the like. Solid carriers include magnesium carbonate, magnesium stearate, sugar, lactose, pectin, dextrin, starch, gelatin, tragacanth, methylcellulose, sodium carboxymethylcellulose, low melting waxes, cocoa butter and the like. Capsules can be made wherein the active compound is inserted into pharmaceutically acceptable capsules as a carrier. The active compounds of this invention can be mixed with pharmaceutically acceptable excipients or can be used in finely divided powder form without excipients for inclusion into capsules. Similarly, cachets are included as are liposomes as known to those skilled in the arts.

Liquid form preparations include solutions, suspensions and emulsions. Examples include water or water-propylene glycol solutions for parenteral injection. Liquid preparations can also be formulated in solution in polyethylene glycol and/or propylene glycol, which may contain water. Aqueous solutions suitable for oral use can be prepared by adding the active component in water and adding suitable colorants, flavors, stabilizing, sweetening, solubilizing and thickening agents as desired. Aqueous suspensions suitable for oral use can be made by dispersing the active component in the oil form within an emulsifier such as TWEEN™-80 as is known in the industry familiar with oil/water emulsions.

However, the compositions of the present invention may also be formulated in other convenient forms for use as a treatment, such as, an injectable solution or suspension, a spray solution or suspension, a lotion, a chewing gum; a lozenge, and a food or snack item. Such items can include any ingestible ingredient, allowing for formulation of the extract of the present invention into food items such as cereals, bars, gumdrops, chewable candies or slowly dissolving lozenges.

In various embodiments, an extract of the invention may be used cosmetically in formulations for such applications, e.g., to reduce or prevent effects associated with oxidative stress, e.g., of skin. The invention provides corresponding cosmetic methods, in which an extract of the invention is applied or administered in a cosmetically acceptable formulation, e.g., in a suitable topical formulation to a site of interest (e.g., an area of skin). Accordingly, the invention also provides cosmetic compositions comprising an extract of the invention and a cosmetically acceptable excipient, carrier or vehicle.

A cosmetically acceptable excipient, carrier or vehicle that may act as a diluent, dispersant or carrier for the extract of the invention and the other materials that may be present in the composition, so as to facilitate their distribution when the composition is applied to the skin. “Cosmetically acceptable carrier”, “cosmetically acceptable excipient” or “cosmetically acceptable vehicle” as used herein refers to one or more compatible solid or liquid fillers, diluents, extenders and the like, which are cosmetically acceptable as defined herein. The term “compatible,” as used herein, means that the components of the compositions of this invention are capable of being commingled with the primary active of the present invention (e.g., the extract described herein), and with each other, in a manner such that there is no interaction that would substantially reduce the efficacy of the composition under ordinary use situations. The type of vehicle utilized in the present invention depends on the type of product desired. The vehicles may also include but are not limited to one or more of organic solvents, thickeners, humectants, oils, silicone oils, water, emulsifiers, liquid or solid emollients, propellants and powders. Such mixtures may take several forms, including but not limited to solutions, dispersions, emulsions (O/W, W/O or W/O/W) such as light creams, lotions, serums, and gels. Powders, such as chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose and ethylene glycol monostearate can be selected.

As used herein, “lotions” are liquid cosmetics, often suspensions or dispersions intended for external application to the body.

As used herein, “creams” are soft cosmetic-type preparations. Creams of the oil-in-water (O/W) type include preparations such as foundation creams, hand creams, shaving creams, and the like. Creams of the water-in-oil (W/O) type include cold creams, emollient creams, and the like. Pharmaceutically, creams are solid emulsions containing suspensions or solutions of active ingredients for external application. Generally, preparations of this type are classified as ointments. Specifically, they belong to the emulsion-type bases.

As used herein, “ointments” are semisolid preparations for external application of such consistency that may be readily applied to the skin. They should be of such composition that they soften, but not necessarily melt, when applied to the body. They serve as vehicles for the topical application of active ingredients and also, function as protectives and emollients for the skin. For many years ointments were limited by definition and use to mixtures of fatty substances. Today, in addition to such oleaginous mixtures, there are ointment preparations possessing the same general consistency but entirely free of oleaginous substances. In many instances, they are emulsions of fatty or wax-like materials with comparatively high proportions of water. These emulsions may be either water-in-oil (W/O) or oil-in-water (O/W) emulsions, depending primarily on the selection of the emulsifying agent. Such semisolid emulsions are also referred to as creams. Creams and ointments containing large amounts of insoluble powders are referred to as pastes. Pastes are usually stiffer and more absorptive than creams and ointments.

For cosmetic applications, a composition of the invention comprising an extract of the invention may further comprise standard cosmetic ingredients, for example those known in the art to be used as moisturizers, stabilizers, preservatives, scents and the like. The type of cosmetic composition may be, for example, skin care cosmetics such as skin lotion, emulsion, cream, and cleansing agents; make-up cosmetics such as lipsticks and foundation. The cosmetics may be in any form without limitation.

The cosmetic compositions of the present invention may further comprise one or more cosmetic agents or dermatological active agents e.g., agents capable of treating or preventing any sign of aging of the skin. The active agents may be chosen, for example, from skin whitening agents, optical brightening agents, sunscreen agents, moisturizers, free-radical scavengers, keratolytic agents, vitamins, anti-elastase and anti-collagenase agents, peptides, fatty acid derivatives, steroids, trace elements, extracts of algae and of planktons, enzymes and coenzymes, flavonoids and ceramides, alpha-hydroxy acids and mixtures thereof, and enhancing agents.

Other cosmetically or dermatologically acceptable agents that may be used in the compositions of the invention include but are not limited to coloring agents (e.g., pigments, dyes, colorants), preservatives, perfumes and fragrances, pulverulent agents, antiperspirants and/or odor absorbers, natural extracts, procyannidol oligomers, urea, caffeine, fillers, keratolytic agents, extracts of algae, fungi, plants, yeasts or bacteria, hydrolysed, partially hydrolysed or unhydrolysed proteins such as enzymes, antibacterial or bactericidal agents e.g., 2,4,4′-trichloro-2′-hydroxydiphenyl ether (triclosan) and 3,4,4′-trichlorocarbanilide (or triclocarban) and azelaic acid, matt-effect agents, for instance fibres, tensioning agents, and mixtures thereof. Amounts of such agents typically range from about 0.0001% to about 20% by weight of the composition.

The composition may be packaged in a suitable container. The choice of container may depend upon the viscosity and intended use of the composition by the consumer. For example, a lotion or fluid cream can be packaged in a bottle or a roll-ball applicator, or a capsule, or a propellant-driven aerosol device or a container fitted with a pump suitable for finger operation. When the composition is a cream, it can simply be stored in a non-deformable bottle or squeeze container, such as a tube or a lidded jar.

In another aspect, the extract and/or compositions comprising the extract of the present invention can be formulated for administration as foods or dietary supplements using one or more consumable carriers. A “consumable carrier” is herein defined as any food, food ingredient, or food additive, or any excipient utilized for tabletting, encapsulation, or other formulation of an active agent for oral administration, whether for human or animal use. For dietary supplements, the extract can be mixed according to methods routine in the art. Dietary supplements can be prepared in a variety of forms including, but not limited to, liquid, powder, or solid pill forms. The extract or composition of the present invention can be administered either alone or in combination with other compounds or extracts where combining compounds or extracts would lead to additive or synergistic effects. The extract and/or composition of the present invention can also be added directly to foods and ingested as part of a normal meal. Various methods are known to those skilled in the art for addition or incorporation of such agents into foods.

The compositions of the invention herein can comprise, consist essentially of, or consist of, the ingredients and components (e.g., the extract) described herein. “Consisting essentially of” as used herein means that the composition may include additional ingredients, but only if the additional ingredients do not materially alter the basic and novel characteristics of the claimed compositions or methods.

In an embodiment the subject is a mammal, in a further embodiment, a human.

The present invention is illustrated in further detail by the following non-limiting examples.

Although the present invention has been described hereinabove by way of specific embodiments thereof, it can be modified, without departing from the spirit and nature of the subject invention as defined in the appended claims. The articles “a,” “an” and “the” are used herein to refer to one or to more than one (i.e., to at least one) of the grammatical object of the article. The terms “including” and “comprising” are used herein to mean, and are used interchangeably with, the phrases “including but not limited to” and “comprising but not limited to.”

### Example 1

### Fucoxanthinol Extraction Process

A quantity of 50 kg of frozen digestive system organs and associated tissue from S. droebachiensis was obtained for use in the extraction process. The material was kept at room temperature for approximately 48 hours in order to completely defrost. The thawed material was homogenized in an industrial blender at 2500 RPM for approximately 30 minutes in order to achieve optimal homogenization.

The 50 kg of homogenized material, still at room temperature, was combined with 50 L of 100% ethanol to give a 1:1 v/v ratio, and allowed to mix in a large vat for 8 to 12 hours with a mixer. The slurry was allowed to settle overnight prior to the centrifugation step. After settling, 50 L of the liquid was pumped from the top of the extraction vat. Fresh ethanol was added to the slurry and the mixing and settling steps were repeated. A total of 3 extractions were conducted.

For each extraction, the pumped liquid supernatant was subjected to centrifugation in 1.4 L quantities at 4060 rpm for 10 minutes. Following centrifugation, the cleared supernatant was placed in a Heidolph rotary evaporator and evaporated at 50° C. under vacuum (50 mbar). Late in the evaporation but prior to the complete evaporation of the ethanol/water mix, the liquid was filtered through a 45 μm filter in order to remove any remaining solids or precipitates.

Following filtration, the remaining liquid was subjected to evaporation again until no further water or solvent was able to be removed. The final liquid which contained the extracted fucoxanthinol had a brown colour and was of an oily consistency.

### Example 2

### Analysis of Concentration Factors

The extract containing fucoxanthinol was tested for fucoxanthin and fucoxanthinol content in order to determine the concentration factors comparing the raw material to the final extract. The concentration factors are outlined below:

TABLE 1
Concentration Factors for Fucoxanthin and Fucoxanthinol

| | | Raw | Final | Concentration |
|---|---|---|---|---|
| Analytes | Units | materials | extract | factors |
| Fucoxanthinol | mg/100 g | 8.4 | 250.0 | 29.8 |
| Fucoxanthin | mg/100 g | 2.8 | 88.4 | 31.6 |
| Fat | g/100 g | 1.9 | 51.7 | 27.2 |

### Analysis Method Summary

Quantitation of fucoxanthin and fucoxanthinol was carried out using HPLC DAD (Extraction column: C18 5 μm 15 cm×4.6 mm; λmax=450 nm) and HPLC-MS (APCI, Mode+, Full Scan) with specific mass for fucoxanthinol 617=[M+H]+, 599=[(M+H)—H₂O]+, 581=[(M+H)-2H₂O]+, 563=[(M+H)-3H₂O]+ and fucoxanthin 659=[M+H]+, 641=[(M+H)—H₂O]+, 623=[(M+H)-2H₂O]+, 605=[(M+H)-3H₂O]+.

Concentration factor=Final extract/Raw materials

### Example 3

### Composition of Final Extract

Samples of the final fucoxanthinol-containing extract were subjected to standardized tests in order to provide a composition analysis. Table 2, below, provides the results for three representative samples.

TABLE 2
Composition of Fucoxanthinol Extract

| Analytes | Units | Sample 1 | Sample 2 | Sample 3 |
|---|---|---|---|---|
| Moisture | g/100 g | 13.9 | 14.9 | 12.4 |
| Protein | g/100 g | 19.5 | 22.3 | 21.3 |
| Fat | g/100 g | 53.9 | 48.8 | 52.3 |
| Ash | g/100 g | 6.6 | 8.8 | 6.3 |
| Carbohydrates | g/100 g | 8.9 | 5.2 | 7.8 |

### Method Summary

OAS-FC01: Determination of Moisture in Foods (AOAC 950.46b(a))
OAS-FC04: Determination of Protein in Foods (AOAC 981.10)
OAS-FC06: Determination of Fat in Foods by Acid Hydrolysis (AOAC 922.06)
OAS-FC02: Determination of Ash in Foods (AOAC 920.153)
Carbohydrates: Calculation
Calories: Calculation

### Example 4

### Microbiological Analysis of Final Extract

In order to ensure that the final extract obtained was safe and suitable for human consumption and use in medicinal compounds or treatments, the extract was subjected to standardized tests to determine the microbiological parameters. The results of three representative samples are provided in Table 3, below.

TABLE 3
Microbiological Analysis of Fucoxanthinol Extract

| Analytes | Units | Sample 1 | Sample 2 | Sample 3 |
|---|---|---|---|---|
| Total Bacteria Count | cfu/g | <10 | <10 | <10 |
| Coliforms | MPN/g | <3 | <3 | <3 |
| E. coli | MPN/g | <3 | <3 | <3 |
| Staphylococcus aureus | cfu/g | <5 | <5 | <5 |
| Salmonella | 25 g | nf | nf | nf |
| Yeasts/Molds | g | <10/<10 | <10/<10 | <10/<10 |

### Method Summary

Tests were performed according to the corresponding Compendium of Analytical Methods, Health Protection Branch and/or AOAC Official Methods
Total Bacteria Count: MFHPB-18
Coliforms: FFA05 as in MFHPB-19
E. coli: FFA05 as in MFHPB-19
Staphylococcus aureus: MFHPB-21
Salmonella: MFHPB-20
Yeasts/Molds: MFHPB-22
nf=not found

### Example 5

### Analysis for Heavy Metals Contained in Final Extract

In order to ensure that the final extract obtained was safe and suitable for human consumption and use in medicinal compounds or treatments, the extract was subjected to standardized tests to determine the amounts of any heavy metals present in the extract. The results of three representative samples are provided in Table 4, below.

TABLE 4
Heavy Metal Analysis of Fucoxanthinol Extract Samples

| Analytes | Units | Sample 1 | Sample 2 | Sample 3 |
|---|---|---|---|---|
| Arsenic | mg/kg | 42.3 | 46.1 | 37.1 |
| Cadmium | mg/kg | <0.02 | <0.02 | <0.02 |
| Lead | mg/kg | <0.02 | <0.02 | <0.02 |
| Mercury | mg/kg | <0.005 | <0.005 | <0.005 |

### Method Summary

Portions of the samples were prepared by Microwave Assisted Digestion in nitric acid. The resulting solutions were analyzed for trace elements by ICP-MS. Mercury was analyzed by Cold Vapour AAS.

What is claimed is:

1. A process for extracting fucoxanthinol from an Echinozoa tissue or organ comprising:
a) providing an Echinozoa tissue or organ;
b) homogenizing said tissue or organ;
c) extracting said fucoxanthinol from the homogenized tissue or organ by one or more solvent extractions, wherein said extraction occurs by thoroughly mixing the homogenized material with the solvent and allowing for settling of the material for at least 8 hours;
d) removing the majority of the liquid portion of the mixture following said settling;
e) evaporating the liquid to remove a portion of the solvent and water mix;
f) filtering the remaining liquid; and
g) performing a final evaporation of the filtered liquid until less than ten percent of the water and solvent is left in the remaining liquid.

2. The process of claim 1 wherein the Echinozoa tissue is obtained in a frozen state, and is allowed to thaw completely prior to the homogenization step (b).

3. The process of claim 1, wherein said tissue or organ is (i) a gonad, (ii) a tissue or organ of the digestive tract, (iii) a viscera, or (iv) any combination of (i) to (iii).

4. The process of claim 1, wherein steps (c) through (d) are repeated between 2 and 5 times in order to maximize the fucoxanthinol content of the final extract.

5. The process of claim 1 or 4, wherein the liquid portion that is removed during step (d) is further subjected to a centrifugation step to remove suspended solids prior to step (e).

6. The process of claim 1, wherein said extraction is performed at a temperature of about 15° C. to about 25° C.

7. The process of claim 1, wherein the solvent used in step (c) is selected from the group consisting of:
a) ethanol;
b) methanol;
c) propanol;
d) n-hexane; and
e) acetone.

8. The process of any one of claims 1 to 7, wherein said Echinozoa is of the Echinoidea class.

9. The process of claim 8, wherein said Echinoidea is selected from the group consisting of:
a) the Diadematoidae family
b) the Arbaciidae family;
c) the Echinidae family;
d) the Echinometridae family; and
e) the Strongylocentrotidae family.

10. The process of claim 9, wherein said Echinoidea is of the Strongylocentrotus genus.

11. The process of claim 10, wherein said Echinoidea of the Strongylocentrotus genus is Strongylocentrotus droebachiensis.

12. The extract of fucoxanthinol prepared according to the process of claim 1.

13. A composition comprising the extract of claim 12 and a carrier.

14. Use of (i) the extract of claim 12, (ii) the composition of claim 13, or (iii) a combination of (i) and (ii), as a medicament.

15. Use of (i) the extract of claim 12, (ii) the composition of claim 13, or (iii) a combination of (i) and (ii), for the preparation of a medicament.

16. Use of (i) the extract of claim 12, (ii) the composition of claim 13, or (iii) a combination of (i) and (ii), as an anti-obesity treatment.

17. A method for preventing or treating a condition in a mammalian subject, said method comprising administering to said subject an effective amount of (i) the extract of claim 12, (ii) the composition of claim 13, or (iii) a combination of (i) and (ii), wherein said condition is selected from the group consisting of:
a) obesity;
b) cancer;
c) diabetes;
d) a neurodegenerative disorder;
e) high blood pressure
f) inflammation; and
g) a condition associated with oxidative stress.
